(19) United States
(12) Patent Application Publication
(10) Pub. No.: US 2017/0290855 A1
(43) Pub. Date: Oct. 12, 2017

(54) TEMPLATE-INDUCED SILICATE-DOPED HYDROXYAPATITE AND THE PREPARATION METHOD

(71) Applicant: ASIA BIOMATERIALS (WUHAN) CO. LTD., Wuhan (CN)

(72) Inventors: Qin YANG, Wuhan (CN); Yingying Du, Wuhan (CN); Jianglin Wang, Wuhan (CN)

(21) Appl. No.: 15/405,975

(22) Filed: Jan. 13, 2017

(30) Foreign Application Priority Data
Apr. 7, 2016 (CN) 201610212667.7

(51) Int. Cl. A61K 33/42; C01B 33/24

(52) U.S. Cl. CPC A61K 33/42; C01B 33/24

(57) ABSTRACT

A silicate-doped hydroxyapatite material has an ordered and directional growth structure. The silicon content in the silicate-doped hydroxyapatite material ranges from 0.1 wt % to 1.6 wt %, and silicon is doped in hydroxyapatite lattices in a form of silicate. A template-induced method for manufacturing the synthetic silicate-doped hydroxyapatite material above.

### CROSS-REFERENCE TO RELATED APPLICATION

This non-provisional application claims priority to and benefit of, under 35 U.S.C. §119(a), Patent Application No. 201610212667.7 filed in P.R. China on Apr. 7, 2016, the entire content of which is hereby incorporated by reference.

### FIELD OF THE INVENTION

The present disclosure relates to the technical field of biomedical material, and particularly to a template-induced silicate-doped hydroxyapatite and the preparation method thereof.

### BACKGROUND OF THE INVENTION

Silicon (Si) is a kind of trace element that exists naturally in various tissues and organs of animal bodies. It has been demonstrated that, silicon is a necessary trace element in new bone growth and cartilage development. Moreover, during the research of bone defect repair, biomaterials containing silicon have multiple advantages compared with traditional calcium-phosphorus (Ca—P) materials. Therefore, if silicon element is incorporated into a bone repair material, the biomedical material can have an excellent biological performance, and silicon element can play its role well during bone defect repair.

Natural bone is mainly constituted by inorganic components and organic components, wherein the inorganic components are mainly low crystalline hydroxyapatite (HA), and the organic components mainly comprise protein and polysaccharide. Hydroxyapatite accounts for about 60 wt % in natural bone, and thus it is the main inorganic component in natural bone tissues. In addition, by virtue of its good biocompatibility and osteoconductivity, hydroxyapatite is widely used in bone repair material field. Due to the important role of silicon element in bone repair as aforesaid, Si-doped hydroxyapatite has been manufactured at present through wet chemical precipitation method, hydrothermal method, and dry method.

Natural bone has a sophisticated and complicated multi level structure both from a microscopic perspective and from a macroscopic perspective, and thus it has an excellent performance of high strength and high toughness. The main reason lies in that, the bio-mineralization procedure during the formation of natural bone is essentially bio-mineralization in-situ by an organic molecule template so as to form organic/inorganic composite material based on progressive assembling of template/hydroxyapatite. Compared with traditional inorganic precipitation synthesis method, the bio-mineralization method has the following three advantages. First, the mineralized product has an ordered structure, and a strength and toughness thereof can both be significantly improved. Second, during bio-mineralization procedure, a preferred crystal growth orientation can be set. Third, there is a mutual action between organic substances and minerals, and minerals are formed during the whole biological metabolism process and participate in the metabolism. Therefore, mineralization and deposition of inorganic components induced by organic molecule template is an effective and important method for manufacturing biomimetic bone repair material.

### SUMMARY OF THE INVENTION

With respect to the technical defect in the prior art, the present disclosure provides a template-induced silicate-doped hydroxyapatite and the preparation method.

The technical solution of the present disclosure will be illustrated in detail hereinafter.

A template-induced silicate-doped hydroxyapatite, wherein the silicate-doped hydroxyapatite material has an ordered and directional growth structure and is synthesized by a template-induced method; and wherein a silicon content in the silicate-doped hydroxyapatite material ranges from 0.1 wt % to 1.6 wt %, and silicon is doped in hydroxyapatite lattices in a form of silicate.

A template-induced method for manufacturing the silicate-doped hydroxyapatite material includes steps of:

(1) providing a silicon source, a template solution, a calcium source solution, and a phosphorus source solution;

(2) mixing the template solution with the calcium source solution and/or the phosphorus source solution;

regulating a pH value of a calcium-containing solution to be neutral; adding the silicon source slowly, dropwise adding a phosphorus-containing solution under a condition of stirring;

regulating a pH value of a reaction system to a needed pH value, then the reaction goes on for a period of time; and

stopping stirring, and collecting a precipitate after the reaction system stays for a period of time so as to obtain a silicate-doped hydroxyapatite material after washing, freezing, and drying,

wherein when the template is a single template which is a non silk fibroin template, the calcium-containing solution is a mixture of the template solution and the calcium source solution, and the phosphorus-containing solution is the phosphorus source solution;

wherein when the template is a single template which is a silk fibroin template, the calcium-containing solution is the calcium source solution, and the phosphorus-containing solution is a mixture of the template solution and the phosphorus source solution;

wherein when the template contains double templates which are both non silk fibroin template, the calcium-containing solution is a mixture of either template of the double templates and the calcium source solution, and the phosphorus-containing solution is a mixture of another template of the double templates and the phosphorus source solution;

wherein when the template contains double templates and one of which is a silk fibroin template, the phosphorus-containing solution is a mixture of the silk fibroin template and the phosphorus source solution, and the calcium-containing solution is a mixture of another template of the double templates and the calcium source solution.

According to the above solution, measuring by calcium element in calcium source solution, measuring by phosphorus element in phosphorus source solution, and measuring by silicon element in silicon source, a molar ratio of calcium element to a sum of phosphorus element and silicon element is about 5:3, and a molar ratio of phosphorus element to silicon element ranges from about 9:1 to 167:1.

According to the above solution, in the template solution, a mass ratio of a template to hydroxyapatite containing silicon is about 3:7.

According to the above solution, the silicon source is tetraethyl orthosilicate.

According to the above solution, the pH value of the reaction system in step (2) is about 7.4.

According to the above solution, the reaction time in step (2) ranges from about 24 h to about 36 h.

According to the above solution, the single template is a molecule template or a composite molecule template, the molecule template is one selected from a group consisting of a protein template, a polysaccharide template, and a synthetic macromolecule template, and the composite molecule template is obtained through cross linking of molecule templates; and the double templates is a combination of two kinds of single template.

According to the above solution, the protein template is one selected from a group consisting of a collagen template, a silk fibroin template, a fibronectin template, a laminin template, and a serum protein template.

According to the above solution, the polysaccharide template is one selected from a group consisting of a hyaluronic acid template, a chondroitin sulfate template, a chitosan template, a sodium alginate template, and a bacterial cellulose template.

According to the above solution, the synthetic macromolecule template is one selected from a group consisting of a synthetic amphiphilic peptide (PAN) template, a cetyltrimethyl ammonium bromide (CTAB) template, a polyacrylic acid (PAA) template, a ethylenediaminetetraacetic acid (EDTA) template, a polystyrene sulfonate (PSS) template, and a polyethylene (PVP) template.

According to the above solution, the composite molecule template is obtained through cross linking of molecule templates by an action of a cross-linking agent or a peptide condensing agent; and the cross linking is a mutual cross linking among a plurality kinds of molecule templates or an internal cross linking among a same kind of molecule templates.

According to the present disclosure, a concentration of the template solution ranges from about 3 mg/ml to about 10 mg/ml, a concentration of the calcium source solution ranges from 0.05 mol/L to 0.1 mol/L, and a concentration of the phosphorus source solution ranges from 0.03 mol/L to 0.06 mol/L. According to the present disclosure, when the template is one selected from a group consisting of the silk fibroin template, the hyaluronic acid template, the chondroitin sulfate template, the sodium alginate template, and composite molecule template, deionized water is used to serve as a solvent. When the template is one selected from a group consisting of the collagen template, the fibronectin template, the chitosan template, and the laminin template, a diluted acid is used to serve as a solvent.

The following beneficial effects can be brought about according to the present disclosure. A template-induced method for manufacturing the silicate-doped hydroxyapatite material is provided. The silicate-doped hydroxyapatite material can be manufactured by a suitable technology and has an ordered and directional growth structure. A silicon content in the silicate-doped hydroxyapatite material ranges from about 0.1 wt % to about 1.6 wt %, and silicon is doped in hydroxyapatite lattices in a form of silicate to replace phosphate. A strength and a toughness of the silicate-doped hydroxyapatite material can be significantly improved, and silicon element can play its role sufficiently in promotion the repair of bone defect. Thus, the silicate-doped hydroxyapatite material is a preferred replace material in bone repair. According to the present disclosure, the manufacturing method is easy to be performed, and the condition thereof can be controlled easily. Therefore, the method has important practical application value.

These and other aspects of the present invention will become apparent from the following description of the preferred embodiment taken in conjunction with the following drawings, although variations and modifications therein may be effected without departing from the spirit and scope of the novel concepts of the disclosure.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIG. 1 shows an X-Ray Diffraction (XRD) diagram of synthetic silicate-doped hydroxyapatite induced by a silk fibroin template according to example 1;

FIG. 2 shows an X-Ray Fluorescence (XRF) diagram of synthetic silicate-doped hydroxyapatite induced by collagen-silk fibroin double templates according to example 3;

FIG. 3 shows an observation result with a high-resolution transmission electron microscopy of synthetic silicate-doped hydroxyapatite crystal induced by chondroitin sulfate-hyaluronic acid double templates according to example 4; and

FIG. 4 shows theoretical models of hydroxyapatite crystal structure and silicon-hydroxyapatite crystal structure, where “a” shows a crystal model of hydroxyapatite, and “b” shows a crystal model of silicon-hydroxyapatite, in which silicate is doped in hydroxyapatite lattices to replace phosphate.

FIG. 5 shows a transmission electron microscope (TEM) image of a silicate-doped hydroxyapatite material according to example 3, where the silicate-doped hydroxyapatite material is synthesized using collagen and silk fibroin double templates.

### DETAILED DESCRIPTION OF THE INVENTION

The present invention is more particularly described in the following examples that are intended as illustrative only since numerous modifications and variations therein will be apparent to those skilled in the art. Various embodiments of the invention are now described in detail. Referring to the drawings, like numbers indicate like components throughout the views. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present invention.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, the terms “comprising”, “including”, “carrying”, “having”, “containing”, “involving”, and the like are to be understood to be open-ended, i.e., to mean including but not limited to.

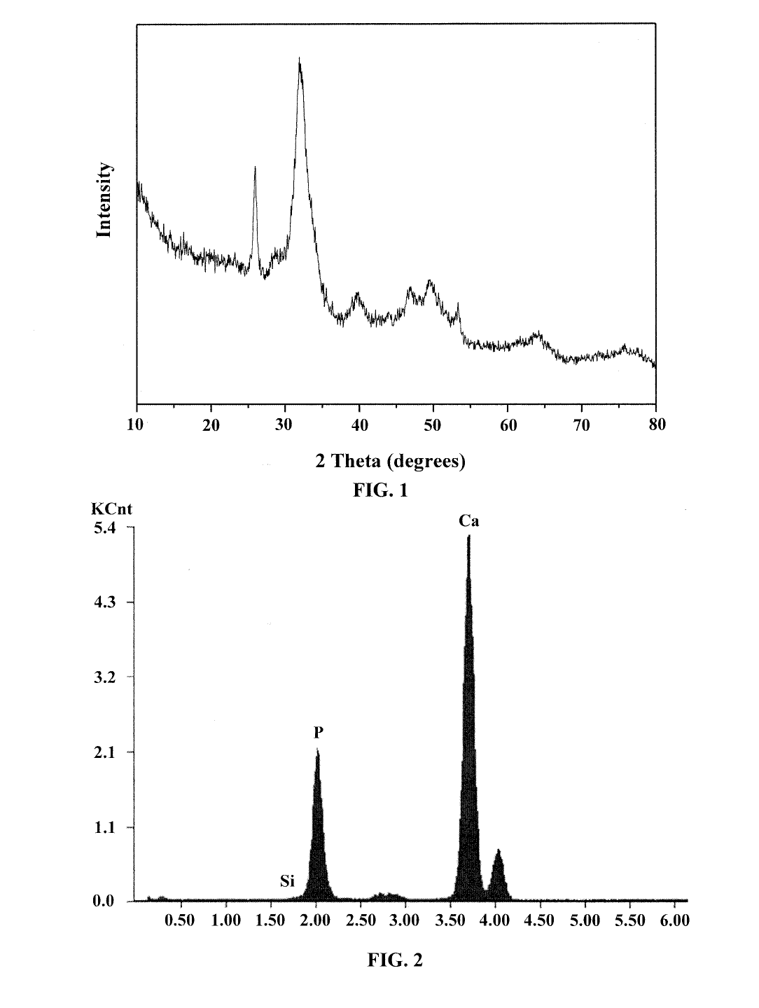

The description will be made as to the embodiments of the present invention in conjunction with the accompanying drawings in FIGS. 1-4. In accordance with the purposes of this invention, as embodied and broadly described herein, this invention, in one aspect, relates to a template-induced silicate-doped hydroxyapatite and a method of making the same.

### EXAMPLE 1

A template-induced silicate-doped hydroxyapatite material can be manufactured through the following method.

(1) Silk fibroin (0.6 g) is dissolved in deionized water (100 ml), and impurities are removed through filtration so as to obtain a silk fibroin solution with a concentration of 6 mg/ml.

(2) Calcium nitrate tetrahydrate (3.29 g) is dissolved in deionized water (200 ml) so as to obtain a calcium source solution.

(3) Diammonium phosphate powder (1.052 g) is dissolved in deionized water (200 ml) so as to obtain a phosphorus source solution; and the silk fibroin solution and the phosphorus source solution are mixed with each other and stirred to form a uniform solution, which stays for 1 hour and a silk fibroin template-phosphorus solution can be obtained.

(4) The calcium source solution is placed in water bath with a constant temperature of 37° C. and stirred, and a pH value of the solution is regulated to 7; and tetraethyl orthosilicate (TEOS) (88.6 μL) is added into the calcium source solution slowly so as to obtain a calcium-silicon solution.

(5) The silk fibroin template-phosphorus solution is dropwise added to the calcium-silicon solution which is stirred constantly at a temperature of 37° C., and a pH value thereof is regulated to be 7.4 by aqueous ammonia; stopping stirring after the reaction goes on for 24 hours, collecting a precipitation after the solution stays for 12 hours, and the precipitation is suction filtrated and washed with deionized water for at least three times; and the powder obtained therein after freezing and drying is the silicate-doped hydroxyapatite powderinduced by a silk fibroin template.

The XRD diagram of the silicate-doped hydroxyapatite powder induced by a silk fibroin template manufactured according to the present example is shown in FIG. 1. As shown by the XRD diagram in FIG. 1, hydroxyapatite characteristic diffraction peaks appear at 2θ being 28° and 32°, which coincide with characteristic peaks shown in hydroxyapatite standard card (PDF card 9-432). Therefore, the main phase of the product is hydroxyapatite. However, compared with the characteristic peaks shown in hydroxyapatite standard card, the characteristic diffraction peaks of the product (silicon-hydroxyapatite) are broadened, which shows that the crystallinity of the material decreases and particle sizes thereof are reduced due to silicon doping. It can be seen through XRF detection that, a silicon content in the silicate-doped hydroxyapatite powder induced by a silk fibroin template is 0.78 wt %.

### EXAMPLE 2

A template-induced silicate-doped hydroxyapatite material can be manufactured through the following method.

(1) Collagen I (0.6 g) is dissolved in acetic acid solution (100 ml) with a concentration of 0.5% (v/v %), and impurities are removed through filtration so as to obtain a collagen I solution with a concentration of 6 mg/ml.

(2) Calcium chloride (1.546 g) is dissolved in deionized water (200 ml) so as to obtain a calcium source solution; and the collagen I solution and the calcium source solution are mixed with each other and stirred to form a uniform solution, which stays for 1 hour and a collagen template-calcium solution can be obtained.

(3) Disodium hydrogen phosphate (1.10 g) is dissolved in deionized water (200 ml), and a phosphorus source solution can be obtained after complete dissolution.

(4) The collagen template-calcium solution is placed in water bath with a constant temperature of 37° C. and stirred, and a pH value of the solution is regulated to 7 with NaOH solution (1 M); and tetraethyl orthosilicate (TEOS) (132.9 μL) is added into the collagen template-calcium solution slowly so as to obtain a calcium-silicon solution.

(5) The phosphorus source solution is dropwise added to the calcium-silicon solution which is stirred constantly, and a pH value of the solution is regulated to 7.4 with NaOH solution (1 M); stopping stirring after the reaction goes on for 24 hours, collecting a precipitation after the solution stays for 12 hours, and the precipitation is suction filtrated and washed with deionized water for at least three times; and the powder obtained therein after freezing and drying is the silicon-doped hydroxyapatite powder induced by a collagen I template.

It can be seen through XRD analysis that, the product obtained in the present example is the silicate-doped hydroxyapatite powder induced by a collagen I template. It can be seen through XRF detection that, a silicon content in the silicate-doped hydroxyapatite powder induced by a collagen I template is 1.095 wt %.

### EXAMPLE 3

A template-induced silicate-doped hydroxyapatite material can be manufactured through the following method.

(1) Collagen I (0.3 g) is dissolved in acetic acid solution (50 ml) with a concentration of 0.5% (v/v %), and impurities are removed through filtration so as to obtain a collagen I solution with a concentration of 6 mg/ml.

(2) Calcium hydroxide (1.032 g) is dissolved in deionized water (200 ml) so as to obtain a calcium source solution; and the collagen I solution and the calcium source solution are mixed with each other and stirred to form a uniform solution, which stays for 1 hour and a collagen template-calcium solution can be obtained.

(3) Silk fibroin (0.3 g) is dissolved in deionized water (50 ml), and impurities are removed through filtration so as to obtain a silk fibroin solution with a concentration of 6 mg/ml.

(4) Ammonium dihydrogen phosphate (1.0456 g) is dissolved in deionized water (200 ml), and a phosphorus source solution can be obtained after complete dissolution; and the silk fibroin solution obtained in step (3) and the phosphorus source solution are mixed with each other and stirred to form a uniform solution, which stays for 1 hour and a silk fibroin template-phosphorus solution can be obtained.

(5) Collagen template-calcium solution is placed in water bath with a constant temperature of 37° C. and stirred, and a pH value of the solution is regulated to 7; and tetraethyl orthosilicate (TEOS) (100.2 μL) is added into the collagen template-calcium solution slowly.

(6) The silk fibroin template-phosphorus solution obtained in step (4) is dropwise added to the collagen template-calcium solution obtained in step (5); the above mixed solution is stirred constantly and a pH value thereof is regulated to 7.4 with aqueous ammonia; stopping stirring after the reaction goes on for 24 hours, collecting a precipitation after the solution stays for 12 hours, and the precipitation is suction filtrated and washed with deionized water for at least three times; and the powder obtained therein after freezing and drying is the silicate-doped hydroxyapatite powder induced by silk fibroin-collagen double templates.

It can be seen through XRD analysis that, the product obtained in the present example is the silicate-doped hydroxyapatite powder induced by silk fibroin-collagen double templates, which shows that the silicon element is doped to hydroxyapatite material successfully. The XRF detection result of the silicate-doped hydroxyapatite powder induced by silk fibroin-collagen double templates according to the present example is shown in FIG. 2. It can be seen from FIG. 2 that, a silicon content in the silicate-doped hydroxyapatite powder induced by silk fibroin-collagen double templates is 0.85 wt %, which is close to theoretical silicon content of 0.9 wt %.

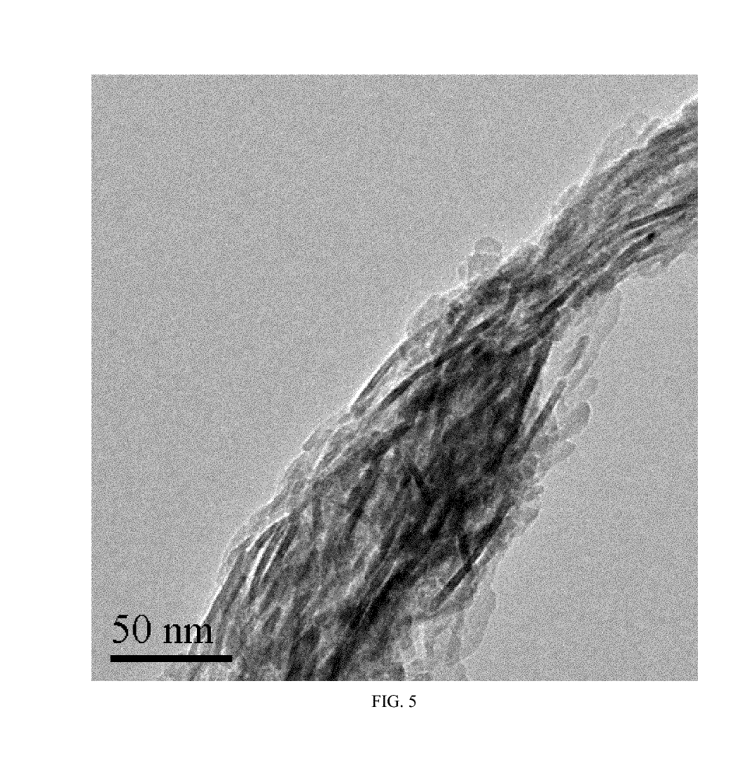

FIG. 5 shows a transmission electron microscope (TEM) image of a silicate-doped hydroxyapatite material according to this example. As shown in FIG. 5, the silicate-doped hydroxyapatite material is synthesized through template induction. Crystals of the silicate-doped hydroxyapatite material grow orderly along a long axis of the template, and the silicate-doped hydroxyapatite material has an ordered and directional growth structure.

### EXAMPLE 4

A template-induced silicate-doped hydroxyapatite material can be manufactured through the following method.

(1) Medical grade chondroitin sulfate (0.3 g) is dissolved in deionized water (50 ml) so as to obtain a chondroitin sulfate template solution with a concentration of 6 mg/ml.

(2) Calcium nitrate tetrahydrate (3.29 g) is dissolved in deionized water (200 ml) so as to obtain a calcium source solution; and the chondroitin sulfate template solution obtained in step (1) and the calcium source solution are mixed with each other and stirred to form a uniform solution, which stays for 1 hour and a chondroitin sulfate template-calcium solution can be obtained.

(3) Hyaluronic acid (0.3 g, Mw: 5 KD) is dissolved in deionized water (50 ml), and impurities are removed through filtration so as to obtain a hyaluronic acid solution with a concentration of 6 mg/ml.

(4) Diammonium phosphate (1.078 g) is dissolved in deionized water (200 ml), and a phosphorus source solution can be obtained after complete dissolution; and the hyaluronic acid solution obtained in step (3) and the phosphorus source solution are mixed with each other and stirred to form a uniform solution, which stays for 1 hour and a hyaluronic acid template-phosphorus solution can be obtained.

(5) Chondroitin sulfate template-calcium solution is placed in water bath with a constant temperature of 37° C. and stirred, and a pH value of the solution is regulated to 7; and tetraethyl orthosilicate (TEOS) (44.3 μL) is added into the chondroitin sulfate template-calcium solution slowly so as to obtain a calcium-silicon solution.

(6) The hyaluronic acid template-phosphorus solution obtained in step (4) is dropwise added to the calcium-silicon solution obtained in step (5); the above mixed solution is stirred constantly and a pH value thereof is regulated to 7.4 with aqueous ammonia; stopping stirring after the reaction goes on for 24 hours, collecting a precipitation after the solution stays for 12 hours, and the precipitation is suction filtrated and washed with deionized water for at least three times; and the powder obtained therein after freezing and drying is the silicate-doped hydroxyapatite powder induced by hyaluronic acid-chondroitin sulfate double templates.

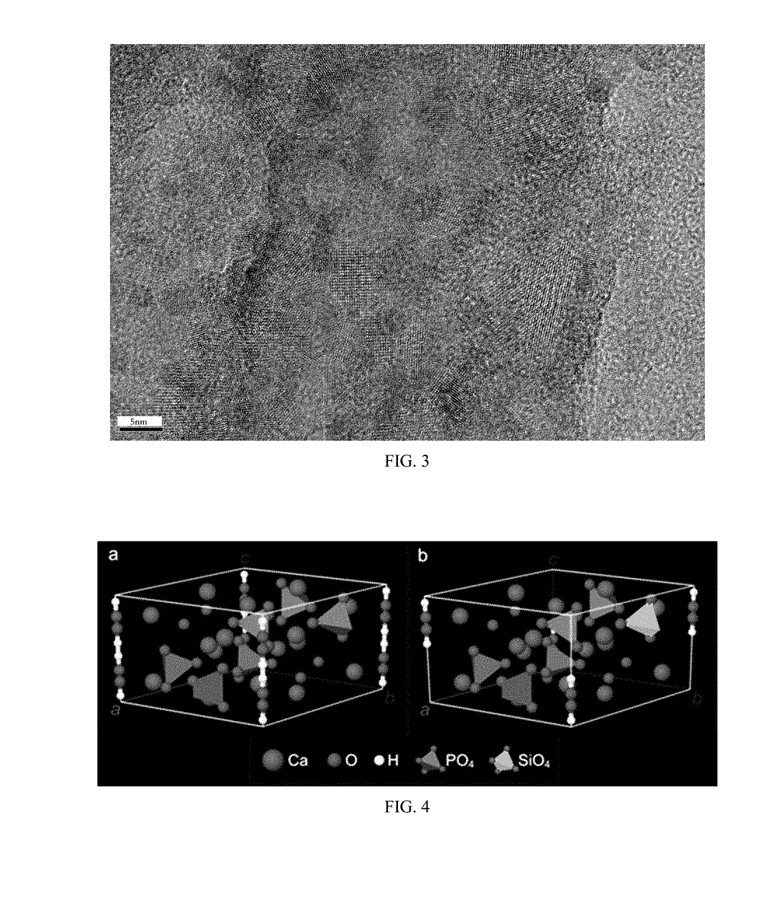

It can be seen through XRD analysis that, the product obtained in the present example is the silicate-doped hydroxyapatite powder induced by hyaluronic acid-chondroitin sulfate double templates. It can be seen through XRF detection that, a silicon content in the silicate-doped hydroxyapatite powder induced by hyaluronic acid-chondroitin sulfate double templates is 0.369 wt %. An observation result with a high-resolution transmission electron microscopy of the silicate-doped hydroxyapatite powder induced by hyaluronic acid-chondroitin sulfate double templates obtained in the present example is shown in FIG. 3. It can be seen from FIG. 3 that, the silicate-doped hydroxyapatite has an ordered and directional growth structure.

### EXAMPLE 5

A template-induced silicate-doped hydroxyapatite material can be manufactured through the following method. (1) Fibronectin (1 g) and bovine serum albumin (0.1 g) are manufactured to be a composite molecule template taking NHS/EDC as catalyst; and the composite molecule template is dissolved in deionized water so as to obtain a composite molecule protein template solution with a concentration of 6 mg/ml.

(2) Calcium nitrate tetrahydrate (3.29 g) is dissolved in deionized water (200 ml) so as to obtain a calcium source solution; and the composite molecule protein template solution obtained in step (1) and the calcium source solution are mixed with each other and stirred to form a uniform solution, which stays for 1 hour and a composite molecule protein template-calcium solution can be obtained.

(3) Disodium hydrogen phosphate (1.00 g) is dissolved in deionized water (200 ml), and a phosphorus source solution can be obtained after complete dissolution.

(4) The composite molecule protein template-calcium solution is placed in water bath with a constant temperature of 37° C. and stirred, and a pH value of the solution is regulated to 7; and tetraethyl orthosilicate (TEOS) (177.3 μL) is added into the solution slowly so as to obtain a calcium-silicon solution.

(5) The phosphorus source solution obtained in step (3) is dropwise added to the calcium-silicon solution obtained in step (3); the above mixed solution is stirred constantly and a pH value thereof is regulated to 7.4 with aqueous ammonia; stopping stirring after the reaction goes on for 24 hours, collecting a precipitation after the solution stays for 12 hours, and the precipitation is suction filtrated and washed with deionized water for at least three times; and the powder obtained therein after freezing and drying is the silicate-doped hydroxyapatite powder induced by a composite molecule protein template.

It can be seen through XRD analysis that, the product obtained in the present example is the silicate-doped hydroxyapatite powder induced by a composite molecule protein template. It can be seen through XRF detection that, a silicon content in the silicate-doped hydroxyapatite powder induced by a composite molecule protein template is 1.532 wt %.

It is obvious that, the above embodiments are described only for better understanding, rather than restricting, the present disclosure. Any person skilled in the art can make amendments or changes based on the contents disclosed herein. It is neither necessary nor feasible to enumerate all of the embodiments. Therefore, the amendments or changes that are readily conceivable based on the technical contents disclosed herein all fall into the protection scope of the present disclosure.

What is claimed is:

1. A silicate-doped hydroxyapatite material synthesized by a template-induced method using a template, wherein the silicate-doped hydroxyapatite material is grown orderly along a long axis of the template to have an ordered and directional growth structure, a content of a silicon in the silicate-doped hydroxyapatite material ranges from about 0.1 wt % to 1.6 wt %, and the silicon is doped in hydroxyapatite lattices of the silicate-doped hydroxyapatite material in a form of silicate.

2. A template-induced method for manufacturing the silicate-doped hydroxyapatite material according to claim 1, comprising:
(1) providing a silicon source, a template solution, a calcium source solution, and a phosphorus source solution;
(2) mixing the template solution with the calcium source solution and/or the phosphorus source solution;
regulating a pH value of a calcium-containing solution to be neutral;
adding the silicon source slowly, and dropwise adding a phosphorus-containing solution under a condition of stirring;
regulating a pH value of a reaction system to a predetermined pH value, so that reaction goes on for a period of time; and
stopping stirring, and collecting a precipitate after the reaction system stays for a period of time so as to obtain a silicate-doped hydroxyapatite material after washing, freezing, and drying,
wherein when the template is a single template which is a non silk fibroin template, the calcium-containing solution is a mixture of the template solution and the calcium source solution, and the phosphorus-containing solution is the phosphorus source solution;
wherein when the template is a single template which is a silk fibroin template, the calcium-containing solution is the calcium source solution, and the phosphorus-containing solution is a mixture of the template solution and the phosphorus source solution;
wherein when the template contains double templates which are both non silk fibroin template, the calcium-containing solution is a mixture of either template of the double templates and the calcium source solution, and the phosphorus-containing solution is a mixture of another template of the double templates and the phosphorus source solution; and
wherein when the template contains double templates and one of which is a silk fibroin template, the phosphorus-containing solution is a mixture of the silk fibroin template and the phosphorus source solution, and the calcium-containing solution is a mixture of another template of the double templates and the calcium source solution.

3. The method according to claim 2, wherein measuring by calcium element in calcium source solution, measuring by phosphorus element in phosphorus source solution, and measuring by silicon element in silicon source, a molar ratio of calcium element to a sum of phosphorus element and silicon element is about 5:3, and a molar ratio of phosphorus element to silicon element ranges from about 9:1 to about 167:1.

4. The method according to claim 2, wherein in the template solution, a mass ratio of a template to hydroxyapatite containing silicon is about 3:7.

5. The method according to claim 2, wherein the silicon source is tetraethyl orthosilicate.

6. The method according to claim 2, wherein the predetermined pH value of the reaction system in step (2) is about 7.4.

7. The method according to claim 2, wherein the reaction time in step (2) ranges from about 24 h to about 36 h.

8. The method according to claim 2, wherein the single template is a molecule template or a composite molecule template, the molecule template is one selected from a group consisting of a protein template, a polysaccharide template, and a synthetic macromolecule template, and the composite molecule template is obtained through cross linking of molecule templates; and wherein the double templates is a combination of two kinds of single template.

9. The method according to claim 8, wherein
the protein template is one selected from a group consisting of a collagen template, a silk fibroin template, a fibronectin template, a laminin template, and a serum protein template;
the polysaccharide template is one selected from a group consisting of a hyaluronic acid template, a chondroitin sulfate template, a chitosan template, a sodium alginate template, and a bacterial cellulose template; and
the synthetic macromolecule template is one selected from a group consisting of a synthetic amphiphilic peptide template, a cetyltrimethyl ammonium bromide template, a polyacrylic acid template, a ethylenediaminetetraacetic acid template, a polystyrene sulfonate template, and a polyethylene template.

10. The method according to claim 8, wherein
the composite molecule template is obtained through cross linking of molecule templates by an action of a cross-linking agent or a peptide condensing agent; and
the cross linking is a mutual cross linking among a plurality kinds of molecule templates or an internal cross linking among a same kind of molecule templates.

11. A method for manufacturing a silicate-doped hydroxyapatite material, comprising:
adjusting pH value of a calcium-containing solution to about pH 7.0;
adding a silicon source slowly to the calcium-containing solution to form a silicon-calcium-containing solution;
adding a phosphorus-containing solution dropwisely to the silicon-calcium-containing solution under stirring to form a reaction solution;
adjusting pH value of the reaction solution to about pH 7.4, such that a reaction is performed in the reaction solution; and
stopping stirring the reaction solution and let the reaction solution stand for a period of time so as to form a precipitate, collecting the precipitate, and washing and lyophilizing the precipitate to obtain the silicate-doped hydroxyapatite material.

12. The method of claim 11, wherein the calcium-containing solution is a mixture of a template solution having a non silk fibroin template and a calcium source solution, and the phosphorus-containing solution is a phosphorus source solution.

13. The method of claim 11, wherein the calcium-containing solution is a calcium source solution, and the phosphorus-containing solution is a mixture of a template solution having a single silk fibroin template and a phosphorus source solution.

14. The method of claim 11, wherein the the calcium-containing solution is a mixture of a first template solution having a first non silk fibroin template and a calcium source solution, and the phosphorus-containing solution is a mixture of a second template solution having a second non silk fibroin template and a phosphorus source solution.

15. The method of claim 11, wherein the calcium-containing solution is a mixture of a first template solution having a first template and a calcium source solution, the phosphorus-containing solution is a mixture of a second template solution having a second template and a phosphorus source solution, and one of the first template and the second template is a silk fibroin template.
